(19) United States
(12) Patent Application Publication
(10) Pub. No.: US 2017/0172922 A1
(43) Pub. Date: Jun. 22, 2017

(54) MICRON TAIWANOFUNGUS CAMPHORATUS LIPOSOME STRUCTURE

(71) Applicant: Hsu-Shen Wu, Taipei (TW)

(72) Inventor: Po-Liang Chen, Taipei (TW)

(21) Appl. No.: 15/412,083

(22) Filed: Jan. 23, 2017

Related U.S. Application Data
(63) Continuation-in-part of application No. 14/186,186, filed on Feb. 21, 2014.

(30) Foreign Application Priority Data
May 15, 2013 (TW) 102209081

(51) Int. Cl. A61K 9/127; A61K 36/07

(52) U.S. Cl. CPC A61K 9/127; A61K 36/07

(57) ABSTRACT

A micron Taiwanofungus camphoratus liposome structure which includes a first and a second liposome shell is provided. The external radius of the first liposome shell is smaller than the internal radius of the second liposome shell, and the second liposome shell encloses the first liposome shell. A first space is defined inside of the first liposome shell, which is for containing at least a first active substance, and a second space, which is for containing at least a second active substance is defined between the inside surface of the second liposome shell and the outside surface of the first liposome shell. A plurality of cup-shaped structures is formed on the outside surface of the second liposome shell. The Taiwanofungus camphoratus liposome structure contains active substances with different effects, such that the storage and transportation of multi-active substances is improved.

### CROSS-REFERENCE TO RELATED APPLICATIONS

This application is a continuation-in-part of U.S. application Ser. No. 14/186,186 filed on Feb. 21, 2014, which claims the priority of Taiwanese patent application No. 102209081 filed on May 15, 2013, the entire contents of which are hereby incorporated by reference.

### TECHNICAL FIELD

The present disclosure relates generally to liposome structure, and more generally to micron Taiwanofungus camphoratus liposome structure for enclosing Taiwanofungus camphoratus and other active substances

### BACKGROUND

The delivering of Taiwanofungus camphoratus is conventionally carried out by oral administration of natural/artificial fruiting body or artificially-fermented mycelia; or by oral administration of water-extracted and ethanol-extracted fruiting bodies. Due to having larger particles, the above-mentioned delivering method will pharmacokinetically lead to a longer digesting and absorbing time, thereby resulting in a nonspecific distribution in human body. Consequently, not only is it unable to satisfy a purpose of centralized-action on target position, but the drug transportation and drug effect are also restricted.

### SUMMARY

The disclosure is directed to a micron Taiwanofungus camphoratus liposome structure which is primarily composed of Phosphatidylcholine (PC), Phosphatidylserine (PS) and phosphatidylinositol (PI). It is capable of enclosing and carrying a plurality of micronized particles of Taiwanofungus camphoratus.

The Taiwanofungus camphoratus liposome structure includes a first liposome shell and a second liposome shell. The external radius of the first liposome shell is smaller than the internal radius of the second liposome shell, and the second liposome shell encloses the first liposome shell. In addition, a first space which is for containing at least a first active substance is defined in the inside of the first liposome shell, and a second space which is for containing at least a second active substance is defined between the inside surface of the second liposome shell and the outside surface of the first liposome shell. A plurality of cup-shaped structures is formed on the outside surface of the second liposome shell.

Another aspect of the disclosure is the first liposome shell and the second liposome shell of the Taiwanofungus camphoratus liposome structure are respectively composed from ethosome and the ethosome includes a plurality of phospholipid molecules and a plurality of ethanol molecules.

Another aspect of the disclosure is the first space of the Taiwanofungus camphoratus liposome structure is for containing at least a first active substance, and the second space is for containing at least a second active substance.

Another aspect of the disclosure is that the cup-shaped structure on the outside surface of the second liposome shell close to specific cancer cell, the cup-shaped structure as receptor to combine with the cancer cell thereof, cause to the Taiwanofungus camphoratus liposome structure is compressed, when Taiwanofungus camphoratus liposome structure in compression to produce disintegration, the interior of at least the plurality of micronized particles of Taiwanofungus camphoratus of Taiwanofungus camphoratus liposome structure scattered respectively on specific cancer cell and to destroy specific cancer cell.

Another aspect of the disclosure is that the Taiwanofungus camphoratus liposome structure is formed by multi-coating process in using a High Pressure Homogenizer.

Another aspect of the disclosure is that the first active substance is not the same as the second active substance, and at least one active substance having distinct components, which is selected from a group consisting of Antroquinonol, Antrocinnamonin A, Antroquinonol B, Antroquinonol D, any combination of group Dehydroeburicoic acid, Dehydrosulphurenic acid, Zhankuic acid A, Zhankuic acid C, Antcin K, and Antcin C.

Another aspect of the disclosure is that the first active substance is selected from a group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts.

Another aspect of the disclosure is that the second active substance is selected from a group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts.

Another aspect of the disclosure is that the phospholipid molecules of the first liposome shell respectively has at least a phospholipid hydrophilic group and at least a phospholipid hydrophobic group, the phospholipid hydrophilic group is located at the inside of the first liposome shell so as to enclose the first active substances from outside to inside, the phospholipid hydrophobic group is located at the outside of the first liposome shell so as to enclose the second active substances from inside to outside.

Another aspect of the disclosure is that the ethanol molecules of the first liposome shell respectively has at least one ethanol hydroxyl and at least one hydrocarbon chain, the ethanol hydroxyl is located at the inside of the first liposome shell so as to enclose the first active substances from outside to inside, the hydrocarbon chain is located at the outside of the first liposome shell so as to enclose the second active substances from inside to outside.

Another aspect of the disclosure is that the phospholipid molecules of the second liposome shell respectively has at least a phospholipid hydrophilic group and at least a phospholipid hydrophobic group, the phospholipid hydrophilic group is located at the inside of the second liposome shell so as to enclose the first active substances from outside to inside, the phospholipid hydrophobic group is located at the outside of the second liposome shell so as to enclose the second active substances from inside to outside.

Another aspect of the disclosure is that the ethanol molecules of the second liposome shell respectively has at least one ethanol hydroxyl and at least one hydrocarbon chain, the ethanol hydroxyl is located at the inside of the second liposome shell so as to enclose the first active substances from outside to inside, the hydrocarbon chain is located at the outside of the second liposome shell so as to enclose the second active substances from inside to outside.

Another aspect of the disclosure is that each of the cup-shaped structures is composed of chitin and beta-cyclodextrin.

It is to be understood that both the foregoing general description and the following detailed description presented below are intended to provide an overview or framework for understanding the nature and character of the disclosure as it is claimed. The accompanying drawings are included to provide a further understanding of the disclosure, and are incorporated into and constitute a part of this specification. The drawings illustrate various embodiments of the disclosure and together with the description serve to explain the principles and operations of the disclosure.

### BRIEF DESCRIPTION OF THE DRAWINGS

FIG. 1 is a partial sectional view of the Taiwanofungus camphoratus liposome structure of the present disclosure;

FIG. 2 is another partial sectional view of the Taiwanofungus camphoratus liposome structure of the present disclosure;

FIG. 3 is a partial sectional view of the Taiwanofungus camphoratus liposome structure of the present disclosure which store active substances;

FIG. 4A is a schematic diagram of phospholipid molecules of ethosome;

FIG. 4B is a schematic diagram of ethanol molecules of ethosome;

FIG. 4C is a schematic diagram of first active substance;

FIG. 4D is a schematic diagram of second active substance;

FIG. 5 is a partial sectional view of the Taiwanofungus camphoratus liposome structure by multi-coating of the present disclosure.

FIG. 6 is another partial sectional view of the Taiwanofungus camphoratus liposome structure by multi-coating of the present disclosure.

FIG. 7 is a diagram of experiment result, showing MA-SNC has obvious effect on suppressing growth of Hep3B liver cancer cells;

FIG. 8A is a photo showing human colorectal cancer cells treated with MA-SNC on day 1;

FIG. 8B is a photo showing human colorectal cancer cells treated with MA-SNC on day 2;

FIG. 8C is a photo showing human colorectal cancer cells treated with MA-SNC on day 3;

FIG. 8D is a photo showing human colorectal cancer cells treated with MA-SNC on day 4;

FIG. 9 shows how apoptosis of human colorectal cancer cells HT-29/SW-480 affected by different concentrations of MA-SNC.

FIG. 10A shows human liver cancer cells treated with high-concentration MA-SNC on day 1;

FIG. 10B shows human liver cancer cells treated with high-concentration MA-SNC on day 2;

FIG. 10C shows human liver cancer cells treated with high-concentration MA-SNC on day 3;

FIG. 10D shows human liver cancer cells treated with high-concentration MA-SNC on day 4;

FIG. 11 shows human liver cancer cells Hep3B are suppressed more by treated with anti-cancer drug Capecitabine+MA-SNC than treated with Capecitabine solely or treated with MA-SNC (3 μg/ml) solely.

### DETAILED DESCRIPTION

Reference is now made in detail to various embodiments of the disclosure, examples of which are illustrated in the accompanying drawings. Whenever possible, the same or like reference numbers and symbols are used throughout the drawings to refer to the same or like parts. The drawings are not necessarily to scale, and one skilled in the art will recognize where the drawings have been simplified to illustrate the key aspects of the disclosure.

The claims as set forth below are incorporated into and constitute part of this Detailed Description.

“Cancer reversion” is a phenomenon which means tumor cells recover to “some normal condition” and are non-threatening so they can peacefully coexist within human body. Taiwanofungus camphoratus is an indigenous fungus in Taiwan, usually cultivated artificially. The ingredients and effects of Taiwanofungus camphoratus are determined by the way of cultivating. The curative effect of Polysaccharides, Triterpenoids and Superoxide Dismutase containing in Taiwanofungus camphoratus have been researched widely.

Tumorigenic HeLa cell surface antigen has an activity of autophosphorylation and is an intestinal alkaline phosphatase (IAP). Taiwanofungus camphoratus and its extracts can inhibit the activity of intestinal alkaline phosphatase in the tumor cell. In addition, it is also demonstrated that Taiwanofungus camphoratus has shown a tendency to improve the performance of tumor suppressor genes.

According to animal experiments, feeding Taiwanofungus camphoratus or its extracts can definitely lead to an effect of tumor suppression. According to the analysis of flow cytometer, the effect of Taiwanofungus camphoratus on tumor cells is not a mechanism of cell cytotoxicity. It is consequently concluded that the effect of Taiwanofungus camphoratus or its extracts on tumor cells is a kind of cancer reversion.

According to the cell assays, it is demonstrated that Taiwanofungus camphoratus and its extracts have obvious effects in inhibiting growth of tumor cells and affecting of virus. In addition, it is also demonstrated that Taiwanofungus camphoratus and extracts thereof can induce cancer reversion.

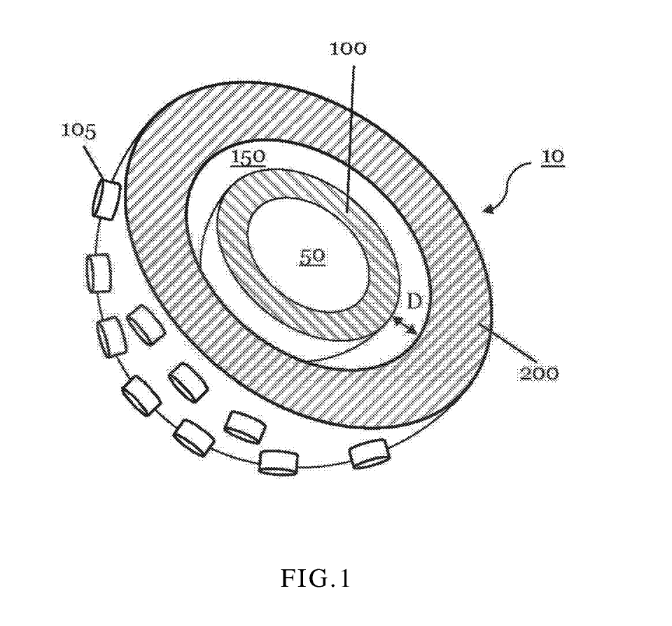

Please refer to FIG. 1, which illustrates a Taiwanofungus camphoratus liposome structure 10, which is also named as Micro Antrocapmphin Smart Nanocarrier (MA-SNC) in the disclosure. Taiwanofungus camphoratus liposome structure 10 is primarily consisted of a first liposome shell 100 and a second liposome shell 200.

Both first liposome shell 100 and second liposome shell 200 are hollow spherical structured, and first liposome shell 100 is located inside of the second liposome shell 200. A first space 50 for containing specific active substances is defined in the inside of first liposome shell 100. The external radius of the first liposome shell 100 is smaller than the internal radius of the second liposome shell 200, the difference between the external radius of the first liposome shell 100 and the internal radius of the second liposome shell 200 is defined as D. A second space 150 is defined between the inside surface of the second liposome shell 200 and the outside surface of the first liposome shell 100.

First space 50 and second space 150 are capable of being used to contain different active substances. Consequently, Taiwanofungus camphoratus liposome structure 10 is capable of enclosing different active substances to be transported and released. The composition of first liposome shell 100 and second liposome shell 200 as well as the active substances contained in the first space 50 and second space 150 will be discussed in following paragraphs.

In addition, a plurality of cup-shaped structures 105 is formed on the outside surface of the second liposome shell 200. Each cup-shaped structure 105 is consisted of chitin and beta-cyclodextrin.

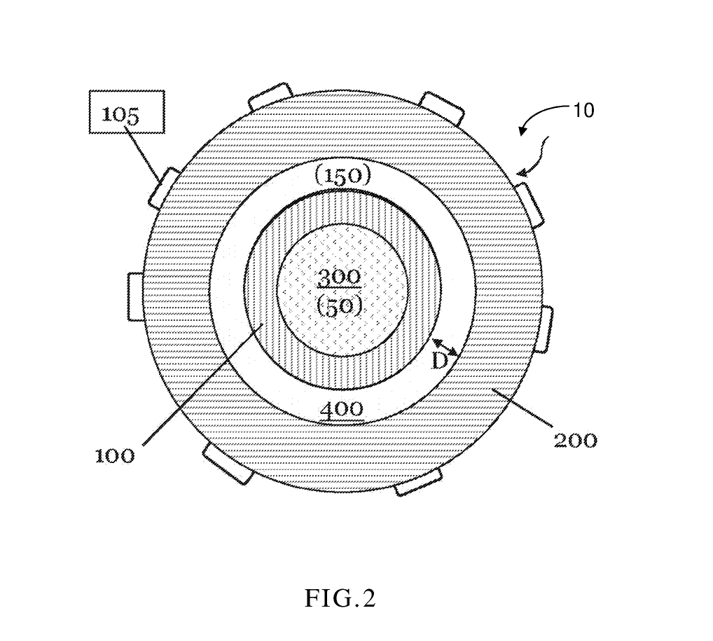

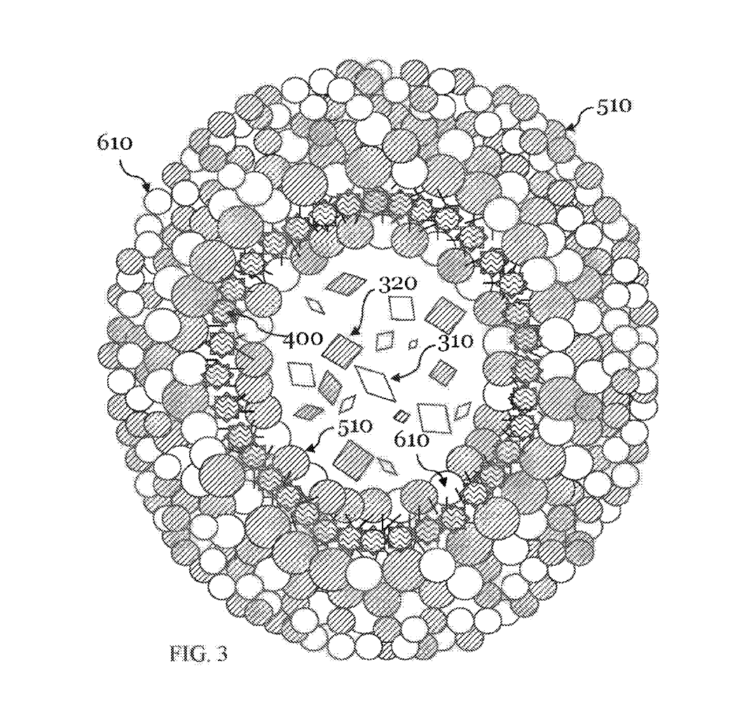

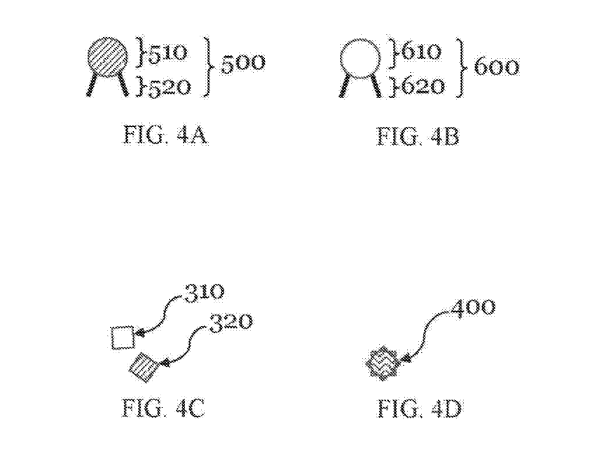

Please refer to FIG. 2, FIG. 3, and FIGS. 4A-4D, in which first liposome shell 100 and second liposome shell 200 are respectively an ethosome with a hollow spherical structure. The ethosome primarily includes a plurality of phospholipid molecules 500 and a plurality of ethanol molecules 600. The ethosome is different from the conventional phytosome. In the ethosome, the contained phospholipid molecules 500 are not combined with the enclosed active substances. While in the conventional phytosome, the phospholipid molecules will be combined with the enclosed active substances and form a new molecule, which remarkably changes the biological property. Consequently, ethosome is more suitable to enclose active substances having different properties than phytosome.

Taiwanofungus camphoratus liposome structure 10 encloses different active substances having different properties. First space 50 of first liposome shell 100 is used for containing first active substances 300, the second space 150 are used to contain second active substances 400. Second active substances 400 include a plurality of micronized particles of Taiwanofungus camphoratus. First active substances 300 are not exactly the same as the second active substances 400. Taiwanofungus camphoratus liposome structure 10 encloses first active substances 300 by first liposome shell 100, second active substances 400 are enclosed between the outside surface of first liposome shell 100 and the inside surface of second liposome shell 200. Further, Taiwanofungus camphoratus liposome structure 10 is capable of being consisted of a plurality of the first liposome shells 100 and the second liposome shell 200.

Taiwanofungus camphoratus liposome structure 10 uses the second space 150 to contain the micronized particles of Taiwanofungus camphoratus, or contain other active substances to achieve the function of sustained release and targeting acting.

Please refer to FIG. 3 and FIGS. 4A-4D, in which first liposome shell 100 is composed of phospholipid molecules 500 and ethanol molecules 600. Each phospholipid molecule 500 has at least a phospholipid hydrophilic group 510 and at least a phospholipid hydrophobic group 520. Phospholipid hydrophilic group 510 is located at the inside of first liposome shell 100 so as to enclose first active substances 300 from outside to inside. Phospholipid hydrophobic group 520 is located at the outside of the first liposome shell 100 so as to enclose second active substances 400 from inside to outside. Ethanol molecules 600 of first liposome shell 100 respectively has at least one ethanol hydroxyl 610 and at least one hydrocarbon chain 620. Ethanol hydroxyl 610 is located at the inside of first liposome shell 100 so as to enclose first active substances 300 from outside to inside. Hydrocarbon chain 620 is located at the outside of first liposome shell 100 so as to enclose second active substances 400 from inside to outside.

Second liposome shell 200 is composed of phospholipid molecules 500 and ethanol molecules 600 as well. Phospholipid molecules 500 of second liposome shell 200 respectively has at least a phospholipid hydrophilic group 510 and at least a phospholipid hydrophobic group 520. Phospholipid hydrophilic group 510 is located at the outside of second liposome shell 200 and phospholipid hydrophobic group 520 is located at the inside of second liposome shell 200, so as to enclose second active substances 400 from outside to inside. Ethanol molecules 600 of second liposome shell 200 respectively has at least one ethanol hydroxyl 610 and at least one hydrocarbon chain 620. Ethanol hydroxyl 610 is located at the inside of second liposome shell 200 and hydrocarbon chain 620 is located at the outside of first liposome shell 100, so as to enclose second active substances 400 from outside to inside.

The previously mentioned first active substance 300 is selected from a group consisted of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts, but not limited thereto. In one example, first active substance 300 has a plurality of hydrophilic components 310 and hydrophobic components 320. Due to being enclosed by ethanol hydroxyl 610 of first liposome shell 100, the biological properties of first active substance 300 can remain when being stored and carried.

The previously mentioned second active substance 400 is selected from a group consisted of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, micronized water-extracted and Ethanol-extracted extracts of artificially-fermented mycelia of Taiwanofungus camphoratus, and seaweed extracts, but not limited thereto. Due to being enclosed between phospholipid hydrophobic group 520 located outside of first liposome shell 100 and hydrocarbon chain 620 located inside of second liposome shell 200, the biological properties of second active substance 400 can be remain when being stored and carried.

Following, through further listed a specific embodiment to description this invention of Taiwanofungus camphoratus liposome structure is formed by multi-coating process, and actual application feasible way; however, one of ordinary skill in the relevant art should be understand clearly. By listed of the specific embodiment and no intent to any form limit this invention of range; this invention certainly not by limited to this, the specific embodiment just, also using other same or equal of function and steps order to reached this invention.

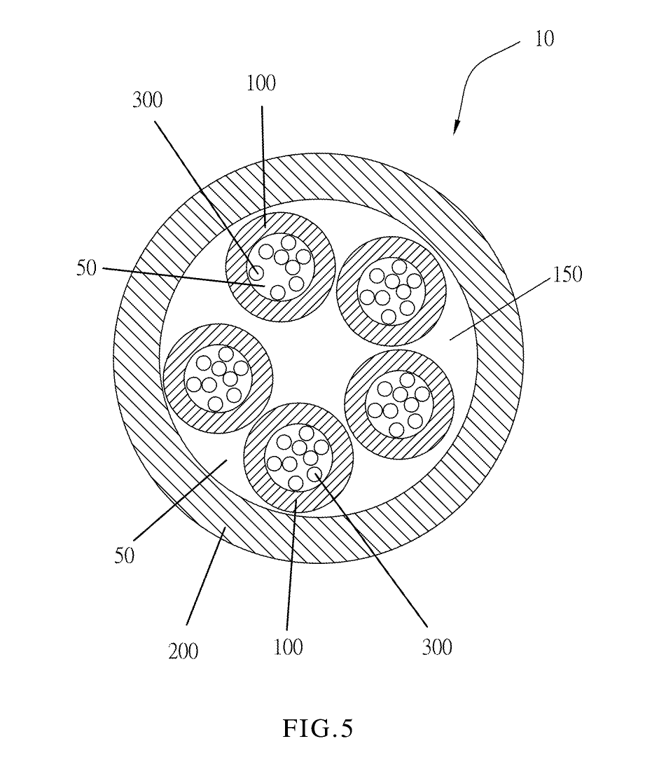

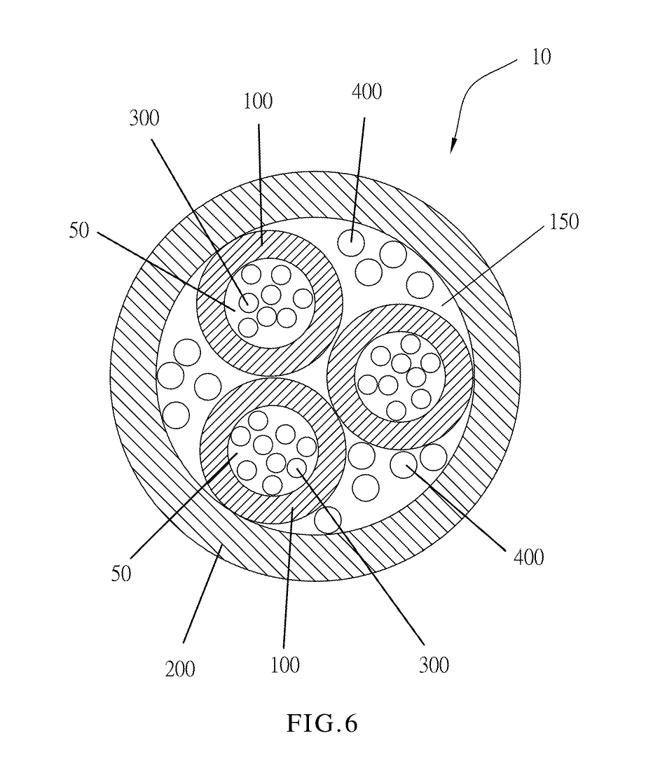

Please refer to FIG. 5 and FIG. 6. First, using a High Pressure Homogenizer, Ethosome is provided to be uniform mixed with grain diameter for about 1 nm-10 nm of particles of the first active substances 300 and surface-active agent in condition that the pressure 500 bar-600 bar, operating temperature 15° C.˜18° C., and the speed 2000 RPM-2500 rpm, to form as FIG. 5 by shows of structure of the first liposome shell 100.

Then, the first liposome shell 100 is provided to be uniform mixed with Ethosome, grain diameter for about 1 nm-10 nm of particles of the first active substances 300 and surface-active agent in condition that the pressure 450 bar˜650 bar, operating temperature 15° C.˜20° C., and the speed 2200 RPM-3000 rpm, to form as FIG. 5 by shows of structure of Taiwanofungus camphoratus liposome structure consisted of the first liposome shell 100, the first active substances 300, the second active substances 400 and the second liposome shell 200.

Taiwanofungus camphoratus liposome structure 10 is formed of multi-coating structure by basically ethosome. First liposome shell 100 and second liposome shell 200 enclose and carry multiple active substances (ie. First active substances 300 and second active substances 400), and keep first active substances 300 and second active substances 400 separated during the storage period. Taiwanofungus camphoratus liposome structure 10 can be designed to release first active substance 300 and second active substance 400 at different locations, resulting in slow-release and centralized action.

In one aspect of the embodiment, the first active substance 300, the second active substance 400 may be independently selected from a group consisting of Antroquinonol, Antrocinnamonin A, Antroquinonol B, Antroquinonol D, Dehydroeburicoic acid, Dehydrosulphurenic acid, Zhankuic acid A, Zhankuic acid C, Antcin K, Antcin C , or any combination thereof, but not limited to the foregoing.

In one aspect of the embodiment, Taiwanofungus camphoratus liposome structure 10 is primarily composed of Phosphatidylcholine (PC), Phosphatidylserine (PS) and phosphatidylinositol (PI). It is capable of enclosing and carrying a plurality of micronized particles of Taiwanofungus camphoratus. The previously mentioned cup-shaped structure 105 can be formed on the outside surface of second liposome shell 200 by the combination between beta-cyclodextrin and Phosphatidylcholine. Be forming a plurality of cup-shaped structures 105 on the outside surface of second liposome shell 200, the amount of Taiwanofungus camphoratus particles being carried can be increased.

When the plurality of cup-shaped structure 105 on the outside surface of second liposome shell 200 as shown in FIG. 1 close to specific cancer cells, the plurality of cup-shaped structure 105 as receptor to combine with the cancer cell thereof, causing the Taiwanofungus camphoratus liposome structure 10 compressed occurred, when Taiwanofungus camphoratus liposome structure 10 in compression to produce disintegration, the interior of at least the plurality of micronized particles of Taiwanofungus camphoratus of Taiwanofungus camphoratus liposome structure 10 scattered respectively on the cancer cells, to destroy the cancer cells.

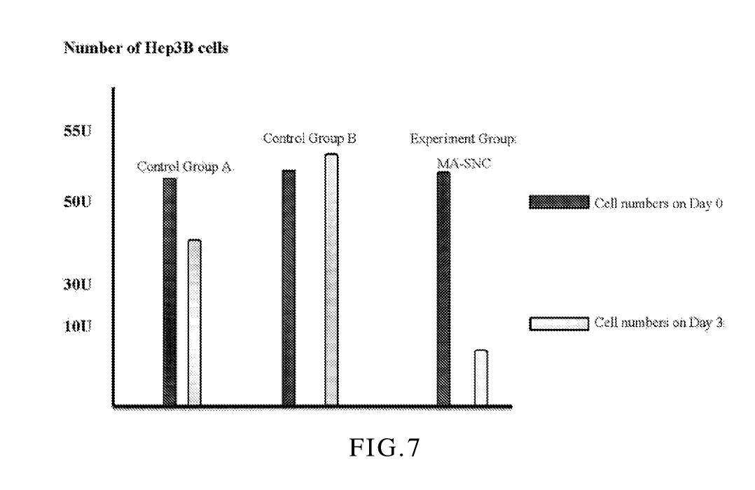

Please refer to FIG. 7, in which the same quantity of culture mediums of liver cancer cells Hep3B have been fed 200 mg per day for three days. Control group A is treated with water-extracted extractions and ethanol-extracted extractions of Taiwanofungus camphoratus. Control group B is treated with water-extracted extractions and ethanol-extracted extractions of commercially available mushroom. Experiment group is tread with MA-SNC. It is shown that MA-SNC has better effect on suppressing growth of Hep3B liver cancer cells than Control group A does.

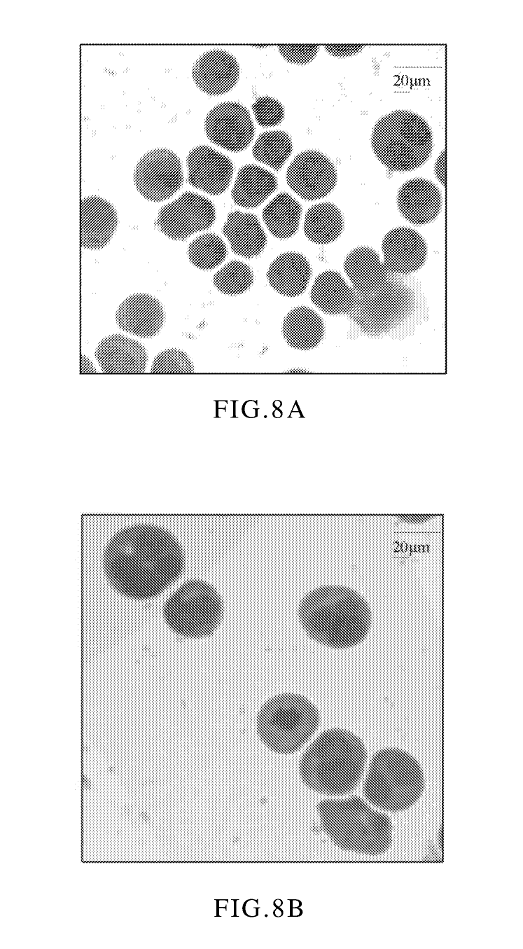

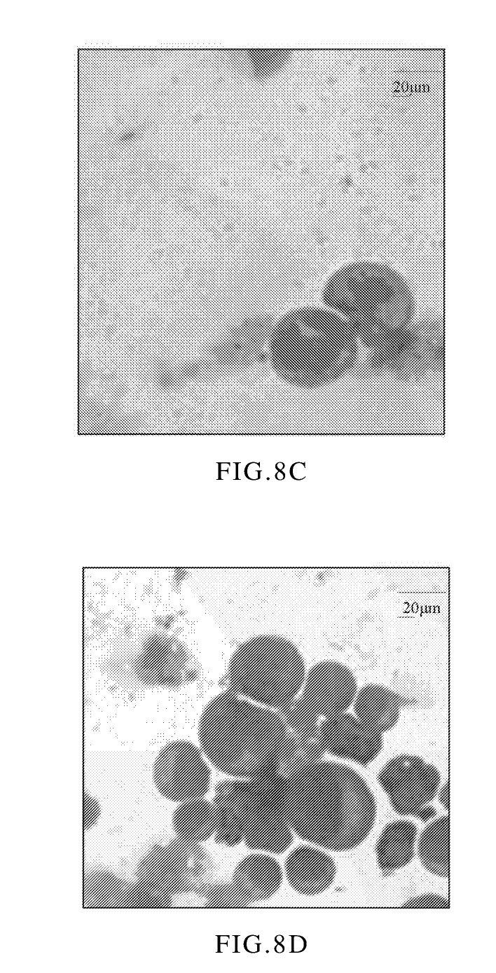

Please refer to FIG. 8A to 8D, respectively showing human colorectal cancer cells treated with MA-SNC from day 1 to day 4. It is shown that cell aggregating phenomenon was obviously reduced, and cancer reversion appeared. MA-SNC has a selective cell cytotoxicity to human colorectal cancer cell HT-29 and SW-480.

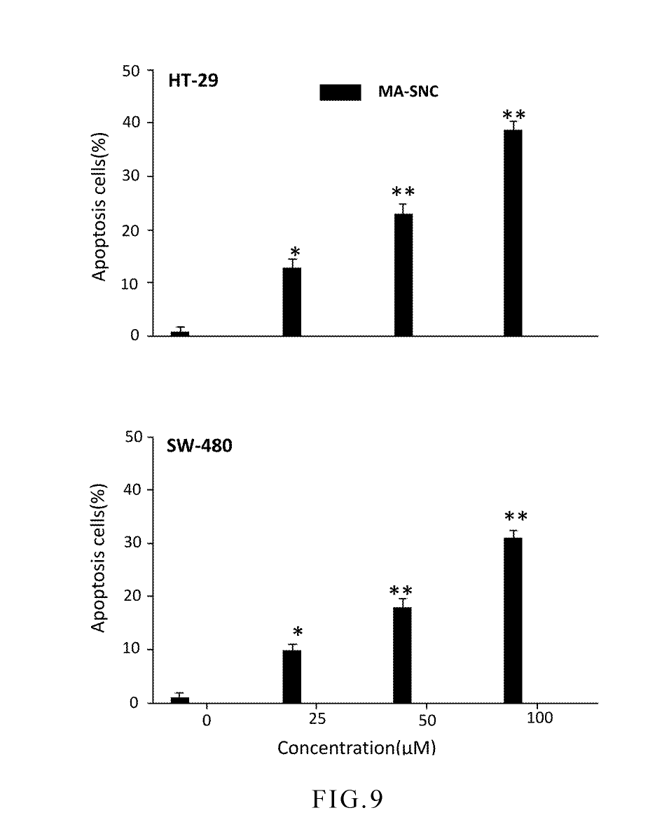

Please refer to FIG. 9, showing how apoptosis of human colorectal cancer cells HT-29/SW-480 is affected by different concentrations of MA-SNC. It is shown that higher concentration of MA-SNC leads to a higher apoptosis.

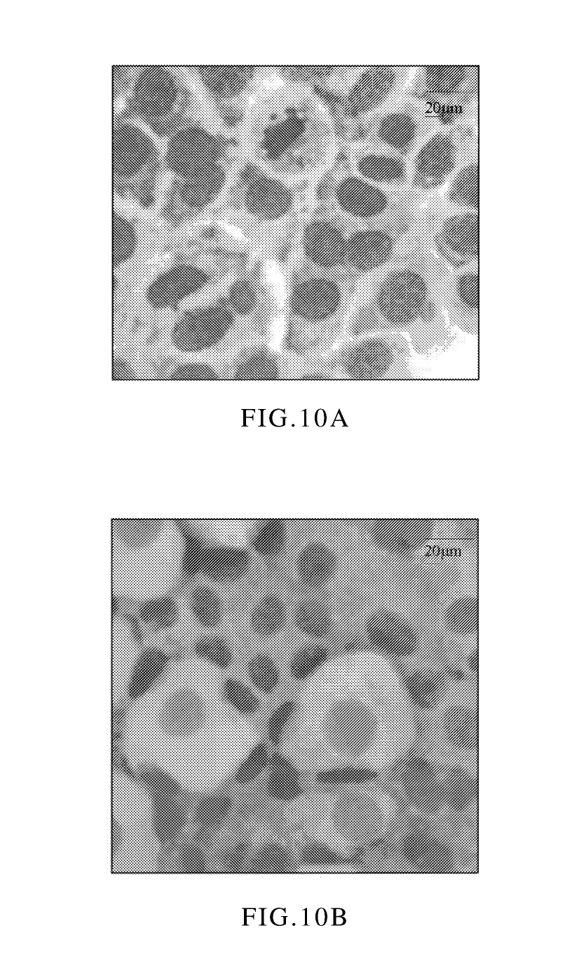

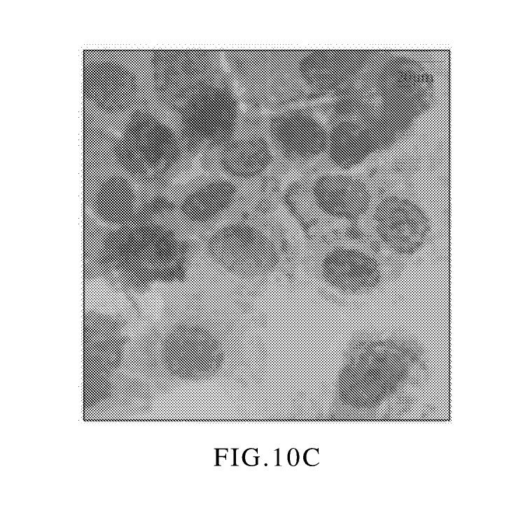

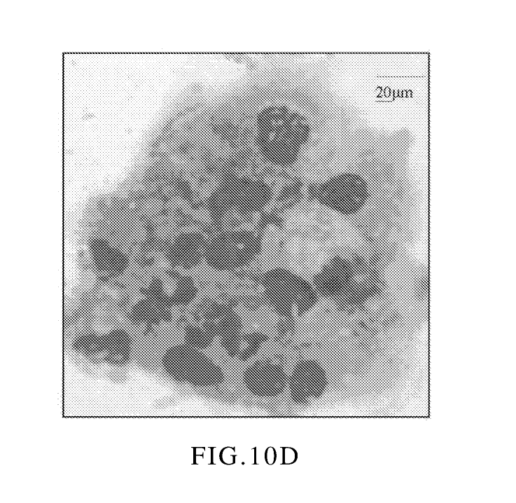

Please refer to FIG. 10A to 10D, respectively showing human liver cancer cells treated with high-concentration MA-SNC from day 1 to day 4. It is shown that MA-SNC can suppress growth of human liver cancer cells HepG2 and lead them to apoptosis.

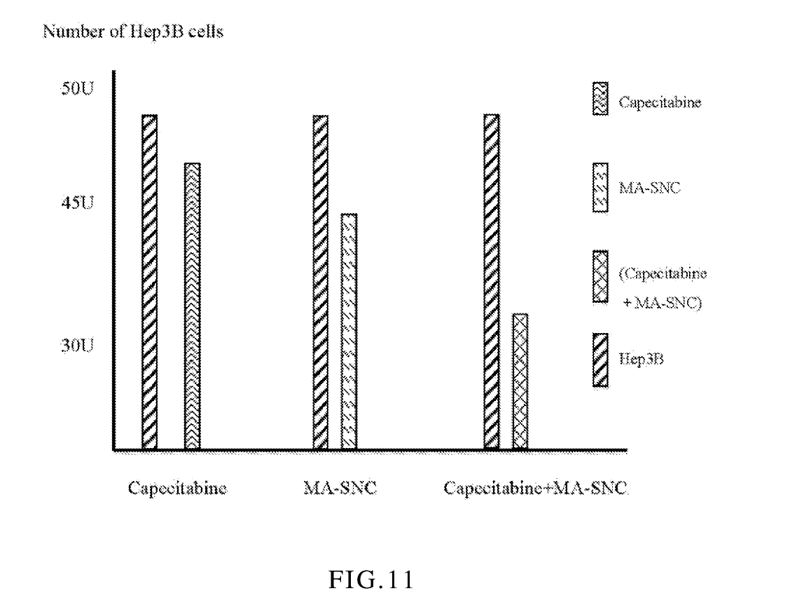

Please refer to FIG. 11, it is demonstrated that human liver cancer cells Hep3B are suppressed more by treatment with anti-cancer drug Capecitabine+MA-SNC than by treatment only with Capecitabine or treated with MA-SNC (3 μg/ml) solely.

It is also demonstrated that MA-SNC can suppress MAPK signal transduction to inhibit metastasis of human breast cancer cells.

It is also demonstrated that MA-SNC is a new selective inhibitor which can suppress Akt/mTOR signal transduction to inhibit growth of threatening human breast cancer cells MDA-MB-231 but shows no cell cytotoxicity to normal mammary cells.

It is also demonstrated that MA-SNC which contains methylantcinate A can suppress of growth of oral cancer OEC-M1 and OC-2, but shows no cell cytotoxicity to normal human gingival fibroblasts cells. MA-SNC containing methylantcinate A can block the combination of epidermal growth factor and receptor to inhibit specialization and spreading of cancer cells.

Taiwanofungus camphoratus liposome structure 10 can be modified to have the following functions:

(1) Response to external factors: for example, response to external factors of temperature, pH value, magnetic force and enzyme, so as to regulate the release of first active substance 300 and second active substance 400 and achieve intelligent sensing and releasing.

(2) Targeting: the ethosome modified with antibody or with specific mark can make the multiple active substances be released at specified location to achieve targeting and centralized action.

To summarize, using Taiwanofungus camphoratus liposome structure as carrier of active substances of micronized Taiwanofungus camphoratus can improve the digesting, absorbing, and distributing of Taiwanofungus camphoratus which is orally taken into the human body.

It will be apparent to those skilled in the art that various modifications and variations can be made to the present disclosure without departing from the spirit and scope of the disclosure. Thus it is intended that the present disclosure cover the modifications and variations of this disclosure provided they come within the scope of the appended claims and their equivalents

What is claimed is:

1. A micron Taiwanofungus camphoratus liposome structure for enclosing and carrying a plurality of micronized particles of Taiwanofungus camphoratus, the Taiwanofungus camphoratus liposome structure comprising:
a plurality of first liposome shells, a first space is defined in each of the inside of the first liposome shell, the first space is for containing at least a first active substance;
a second liposome shell, enclosing the first liposome shell, external radius of the first liposome shell is smaller than internal radius of the second liposome shell,
a second space is defined between the inside surface of the second liposome shell and the outside surface of the first liposome shell, the second space is for containing at least a second active substance; and
a plurality of cup-shaped structures is formed on the outside surface of the second liposome shell.

2. The micron Taiwanofungus camphoratus liposome structure according to claim 1, wherein the first liposome shell and the second liposome shell are respectively composed of ethosome which comprising a plurality of phospholipid molecules and a plurality of ethanol molecules.

3. The micron Taiwanofungus camphoratus liposome structure according to claim 1, wherein the cup-shaped structure on the outside surface of the second liposome shell close to specific cancer cell, the cup-shaped structure as receptor to combine with the cancer cell thereof, cause to the Taiwanofungus camphoratus liposome structure is compressed, when the Taiwanofungus camphoratus liposome structure in compression to produce disintegration, the interior of at least the plurality of micronized particles of Taiwanofungus camphoratus of the Taiwanofungus camphoratus liposome structure scattered respectively on specific cancer cell and to destroy specific cancer cell.

4. The micron Taiwanofungus camphoratus liposome structure according to claim 1, wherein the Taiwanofungus camphoratus liposome structure is formed by multi-coating process in using a High Pressure Homogenizer.

5. The micron Taiwanofungus camphoratus liposome structure according to claim 1, wherein the first active substance is not the same as the second active substance, and at least one active substance having distinct components, which is selected from a group consisting of Antroquinonol, Antrocinnamonin A, Antroquinonol B, Antroquinonol D, any combination of group Dehydroeburicoic acid, Dehydrosulphurenic acid, Zhankuic acid A, Zhankuic acid C, Antcin K, and Antcin C.

6. The micron Taiwanofungus camphoratus liposome structure according to claim 1, wherein the first active substance is selected from a group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, and micronized water-extracted and Ethanol-extracted extracts of artificially-fermented mycelia of Taiwanofungus camphoratus.

7. The micron Taiwanofungus camphoratus liposome structure according to claim 1, wherein the second active substance is selected from a group consisting of micronized water-extracted and Ethanol-extracted fruiting bodies of natural Taiwanofungus camphoratus, micronized water-extracted and Ethanol-extracted fruiting bodies of basswood cultivated Taiwanofungus camphoratus, and micronized water-extracted and Ethanol-extracted extracts of artificially-fermented mycelia of Taiwanofungus camphoratus.

8. The micron Taiwanofungus camphoratus liposome structure according to claim 7, wherein the phospholipid molecules of the first liposome shell respectively has at least a phospholipid hydrophilic group and at least a phospholipid hydrophobic group, the phospholipid hydrophilic group is located at the inside of the first liposome shell so as to enclose the first active substances from outside to inside, the phospholipid hydrophobic group is located at the outside of the first liposome shell so as to enclose the second active substances from inside to outside.

9. The micron Taiwanofungus camphoratus liposome structure according to claim 7, wherein the ethanol molecules of the first liposome shell respectively has at least one ethanol hydroxyl and at least one hydrocarbon chain, the ethanol hydroxyl is located at the inside of the first liposome shell so as to enclose the first active substances from outside to inside, the hydrocarbon chain is located at the outside of the first liposome shell so as to enclose the second active substances from inside to outside.

10. The micron Taiwanofungus camphoratus liposome structure according to claim 7, wherein the phospholipid molecules of the second liposome shell respectively has at least a phospholipid hydrophilic group and at least a phospholipid hydrophobic group, the phospholipid hydrophilic group is located at the inside of the second liposome shell so as to enclose the first active substances from outside to inside, the phospholipid hydrophobic group is located at the outside of the second liposome shell so as to enclose the second active substances from inside to outside.

11. The micron Taiwanofungus camphoratus liposome structure according to claim 7, wherein the ethanol molecules of the second liposome shell respectively has at least a ethanol hydroxyl and at least a hydrocarbon chain, the ethanol hydroxyl is located at the inside of the second liposome shell so as to enclose the first active substances from outside to inside, the hydrocarbon chain is located at the outside of the second liposome shell so as to enclose the second active substances from inside to outside.

12. The micron Taiwanofungus camphoratus liposome structure according to claim 1, wherein each of the cup-shaped structures is composed of chitin and beta-cyclodextrin.
